(19) United States
(12) Patent Application Publication
(10) Pub. No.: US 2008/0103220 A1
(43) Pub. Date: May 1, 2008

(54) SYNTHETIC FUEL PRODUCTION USING COAL AND NUCLEAR ENERGY

(75) Inventors: Robert S. Cherry, Idaho Falls, ID (US); Richard A. Wood, Idaho Falls, ID (US)

Correspondence Address: BATTELLE ENERGY ALLIANCE, LLC, P.O. BOX 1625, IDAHO FALLS, ID, 83415-3899, US

(73) Assignee: BATTELLE ENERGY ALLIANCE, LLC, Idaho Falls, ID (US)

(21) Appl. No.: 11/552,604

(22) Filed: Oct. 25, 2006

(51) Int. Cl. B01J 8/00; C07C 27/06

(52) U.S. Cl. 518/702; 422/189; 422/234; 518/705

(57) ABSTRACT

Oxygen and hydrogen produced in an electrolysis process are used with a carbon-to-liquid hydrocarbon production process to improve the conversion of carbon in coal to synthesis gas products subsequently converted to liquid hydrocarbons. Carbon-containing tail gases and pollutants in a coal-to-liquid hydrocarbon production process may also be recycled to a coal gasification process to improve the conversion of carbon to liquid hydrocarbons and to reduce carbon-based pollutants from the coal-to-liquid hydrocarbon production process.

### GOVERNMENT RIGHTS

The United States Government has certain rights in this invention pursuant to Contract No. DE-AC07-05-ID14517 between the United States Department of Energy and Battelle Energy Alliance, LLC.

### BACKGROUND OF THE INVENTION

1. Field of the Invention

The present invention relates to processes and systems for converting carbonaceous feedstocks to liquid hydrocarbons and liquid fuel products and, more particularly, to the use of alternative energy sources and recycling processes to provide improved recovery in coal-to-liquid processes and systems.

2. State of the Art

Processes for producing liquid fuel products from coal are well known. One of the more common processes involving the conversion of coal to liquid hydrocarbon fuels involves Fischer-Tropsch processes whereby synthesis gas, or syngas, is converted into liquid fuel products of various forms. Synthesis gas for use in the Fischer-Tropsch process may be produced by the gasification of coal which produces carbon monoxide and hydrogen.

An example of a conventional coal-to-liquid hydrocarbon process is illustrated in the process flow diagram of FIG. 1. The coal-to-liquid hydrocarbon (CTL) process 100 may include a number of sub-processes occurring within the CTL process 100. For example, a conventional CTL process 100 may include a coal gasification process 110, a gas and heat recovery process 120, a gas cleanup process 130, a Fischer-Tropsch process 160, and a heat recovery and power generation process 190. Each of the sub-processes illustrated in FIG. 1 is separated by dashed lines.

The coal gasification process 110 of the CTL process 100 includes a dryer 112, a pulverizer 114, an entrained flow gasifier 116, and an air separation unit 118. Coal 111 for use in the coal gasification process 110 is fed to one or more dryers 112 to reduce moisture in the coal 111. Moisture extracted from the coal 111 may be discharged from the dryers 112. A dryer 112 may include a heat exchanger or other conventional drying process typically used with CTL processes 100. The dried coal 113 is fed to a pulverizer 114 where the dried coal 113 is pulverized, crushed, or otherwise reduced to a sufficient size for coal gasification. The pulverized coal 115 is fed to the entrained flow gasifier 116 where the coal is gasified. Air 119 fed to the air separation unit 118, such as a cryogenic air separation unit, is converted into nitrogen (N₂) and oxygen (O₂). The nitrogen is purged as nitrogen stream 118b while the oxygen stream 118a is fed to the entrained flow gasifier 116.

The pulverized coal 115 fed to the entrained flow gasifier 116 is gasified with oxygen from the oxygen stream 118a in the presence of steam 50 fed to the entrained flow gasifier 116. Boiler feed water (BFW) 52 may also be fed to the entrained flow gasifier to absorb heat produced in the gasification process, forming a medium pressure steam (MPS) 54 which may be removed from the entrained flow gasifier 116 and used elsewhere in the CTL process 100. The combustion, pyrolysis, and gasification of the pulverized coal 115 fed to the entrained flow gasifier 116 produces a syngas product 116a and a slag 116b. The syngas product 116a is removed from the entrained flow gasifier 116 and the coal gasification process 110 and is fed to a gas and heat recovery process 120. Slag 116b is removed from the entrained flow gasifier 116 and is disposed of or otherwise utilized according to conventional methods.

The syngas product 116a from the entrained flow gasifier 116 includes carbon monoxide (CO), hydrogen (H₂), and other gas products. The hot syngas product 116a is fed to a syngas cooler 122 in the gas and heat recovery process 120. The syngas cooler 122 cools the hot syngas product 116a and produces a cool syngas product 123 which is withdrawn from the syngas cooler 122. The cool syngas product 123 is then fed to a gas cleanup process 130.

The hot syngas product 116a fed to the syngas cooler 122 may also be combined with a quench gas 143 from the gas cleanup process 130, the quench gas having been fed through one or more compressors 124.

Boiler feed water 52 may also be fed to the syngas cooler 122. The boiler feed water 52 absorbs heat within the syngas cooler 122 producing steam, such as high pressure steam 56 and medium pressure steam 54. High pressure steam 56 produced in the syngas cooler 122 is discharged. High pressure steam 56 may also be fed to the sour shift reactor 138 of the gas cleanup process 130. Medium pressure steam 54 produced in the syngas cooler 122 may be recovered and used elsewhere in the CTL process 100.

The gas cleanup process 130 is used to remove pollutants and other unwanted products from the cool syngas product 123 fed to the gas cleanup process 130. The gas cleanup process 130 includes a cyclone 132, a water scrubber 134, a black water system 136, a sour shift reactor 138, a heat exchanger 140, a condenser 142, and an activated carbon bed 144. The gas cleanup process 130 also includes equipment for removing hydrogen sulfide (H₂S) and carbon dioxide (CO₂) from synthesis gas.

Cool syngas product 123 fed to the gas cleanup process 130 is fed to a cyclone 132 to remove flyash 133 and other particulates from the cool syngas product 123. The cool syngas product 123 is then fed to a water scrubber 134 with water 60. The water scrubber 134 removes pollutants and other impurities from the cool syngas product 123 and discharges the removed pollutants and other impurities with water 60 in a waste water stream 134a. A scrubbed syngas product 135 is split into a first scrubbed syngas stream 135a that is fed to the sour shift reactor 138 and a second scrubbed syngas stream 135b that bypasses the sour shift reactor 138 and is combined with the shifted syngas product 138a from the sour shift reactor 138. The first scrubbed syngas stream 135a fed to the sour shift reactor 138 is combined with high pressure steam 56 fed to the sour shift reactor 138 to produce desired ratios of hydrogen and carbon monoxide for the syngas being produced. The shift of the hydrogen and carbon monoxide ratios in the shifted syngas product 138a from the sour shift reactor 138 may be manipulated such that the combination of the shifted syngas product 138a with the second scrubbed syngas product 135b produces a syngas product 139 having a desired ratio of hydrogen and carbon monoxide for the Fischer-Tropsch process 160.

The waste water stream 134a is fed to a black water treatment system 136 for treatment of the waste water stream 134a according to conventional methods.

The syngas product 139 is fed to a heat exchanger 140 that cools the syngas product 139. Boiler feed water 52 fed to the heat exchanger 140 may be converted into medium pressure steam 54, which may be used in other portions of the CTL process 100.

The cooled syngas product 141 from the heat exchanger 140 is fed to a condenser 142 where water 60 in the cooled syngas product 141 is condensed and removed from the cooled syngas product 141. The water 60 may be fed to the water scrubber 134 in the gas cleanup process 130.

The syngas product 143 from the condenser 142 is fed to an activated carbon bed 144. The activated carbon bed 144 removes unwanted mercury pollutants from the syngas product 143. A portion of the syngas product 143 exiting the activated carbon bed 144 may be removed and fed to the compressor 124 of the gas and heat recovery process 120 and combined with the syngas product 116a as feed stock to the syngas cooler 122. The remainder of the syngas product 143 is fed to a Rectisol process 146 to further remove hydrogen sulfide (H₂S) and carbon dioxide (CO₂) from the syngas product 143.

The Rectisol process 146 removes hydrogen sulfide and carbon dioxide from the syngas product 143, producing a first product stream 146a containing hydrogen sulfide and carbon dioxide, a second product stream 146b containing carbon dioxide, a purge gas 146c, and a syngas product stream 147.

The first product stream 146a can be fed to a Claus process 148 for removal of sulfur 148a from the gaseous hydrogen sulfide in the first product stream 146a. The Claus tail gases 148b are fed to a SCOT process 150 for further treatment before being discharged as a SCOT process purge gas 150a.

The second product stream 146b of carbon dioxide is compressed in a first compressor 152 and then fed to a triethylene glycol (TEG) dehydration process 154 where water is removed from the second product stream 146b. Water removed from the second product stream 146b may be used elsewhere in the CTL process 100. Carbon dioxide gas from the TEG process 154 is fed to a compressor 156 where it is cooled to form liquid carbon dioxide 157. The liquid carbon dioxide 157 is pumped to a storage tank or other process for further use.

The purge gas 146c from the Rectisol process 146 is released into the environment. The Rectisol process 146, Claus process 148, SCOT process 150, and TEG dehydration process 154 are well known processes conventionally used to remove sulfur and carbon dioxide pollutants from CTL processes 100.

The syngas product stream 147 is fed to a Fischer-Tropsch process 160 for conversion of the syngas product stream 147 into liquid fuel products. The Fischer-Tropsch process 160 illustrated in FIG. 1 includes a first Fischer-Tropsch reactor 162, a first heat exchanger 164, a first separation unit 166, a compressor 168, a second Fischer-Tropsch reactor 172, a second heat exchanger 174, a second separation unit 176, a hydrocracker 170, a hydrogen separation membrane 178, a second compressor 180, a tank 182, and distillation columns 184.

The syngas product stream 147 from the gas cleanup process 130 contains hydrogen (H₂) and carbon monoxide (CO). In the Fischer-Tropsch process 160, the syngas product stream 147 is fed to the first Fischer-Tropsch reactor 162 where the hydrogen and carbon monoxide in the syngas product stream 147 are converted into liquid fuel products through a catalysis reaction, such as Reaction 1:

(2n+1) H₂+nCO→CₙH₂ₙ₊₂+nH₂O (1),

where n may be between about 1 and about 42, although n may also be greater than 42. For example, for light hydrocarbons n may be between about 1 and about 7, for intermediate hydrocarbons n may be from about 8 to about 13, and for heavy hydrocarbons n may be above about 14.

A first hydrocarbon stream 163 containing both liquid fuel products and gas from the first Fischer-Tropsch reactor 162 is cooled in the first heat exchanger 164. Boiler feed water 52 fed to the first heat exchanger 164 absorbs heat from the first hydrocarbon stream 163, producing medium pressure steam 54 that is purged for use in other processes. The cooled first hydrocarbon stream 163 is fed to the first separation unit 166 where various constituents of the first hydrocarbon stream 163 are separated. Syngas 167 remaining in the first hydrocarbon stream 163 is separated by the first separation unit 166 and purged to be fed to the second Fischer-Tropsch reactor 172. The liquid fuel products in the first hydrocarbon stream 163 are separated by the first separation unit 166 into two hydrocarbon streams: a first light hydrocarbon product 166a containing intermediate distillates and light hydrocarbons, and a first heavy hydrocarbon product 166b containing heavy hydrocarbons. The first separation unit 166 also removes water 60 from the first hydrocarbon stream 163 and purges the water 60 from the other products.

Syngas 167 from the first separation unit 166 is compressed in the compressor 168 and is then fed to the second Fischer-Tropsch reactor 172 where the syngas 167 is catalytically reacted to convert the hydrogen and carbon monoxide in the syngas 167 into liquid fuel products. For example, the syngas 167 may be converted into liquid fuel products in accordance with Reaction 1. A second hydrocarbon stream 173 from the second Fischer-Tropsch reactor 172 is cooled in the second heat exchanger 174 and fed to the second separation unit 176. Boiler feed water 52 fed to the second heat exchanger 174 absorbs heat from the second hydrocarbon stream 173, producing medium pressure steam 54 that is purged for use in other processes or other parts of the CTL process 100.

The second separation unit 176 separates the second hydrocarbon stream 173 into its various constituents. Water 60 separated from the second hydrocarbon stream 173 is purged from the second separation unit 176 for use elsewhere. Liquid fuel products in the second hydrocarbon stream 173 are separated into two hydrocarbon streams: a second light hydrocarbon product 176a containing intermediate distillates and light hydrocarbons, and a second heavy hydrocarbon product 176b containing heavy hydrocarbons. The tail gases 177 separated from the second hydrocarbon stream 173 in the second separation unit 176 are fed to the hydrogen separation membrane 178 where the hydrogen (H₂) 179 in the tail gases 177 is removed.

Heavy hydrocarbons produced in the Fischer-Tropsch process 160 are converted to light hydrocarbons or liquid fuel products in the hydrocracker 170. The first heavy hydrocarbon product 166b and the second heavy hydrocarbon product 176b produced by the Fischer-Tropsch reactors are mixed together to form a heavy hydrocarbon stream 169 that is fed to the hydrocracker 170. Heavy hydrocarbons 185 from the distillation column 184 are also fed to the hydrocracker 170. Hydrogen 179, separated from the tail gases 177 by the hydrogen separation membrane 178, is pressurized or compressed in compressor 180 before being fed to the hydrocracker 170 to facilitate the hydrocracking of the heavy hydrocarbons to produce a hydrocarbon product 171 of lighter hydrocarbons or liquid fuel products from the hydrocracker 170.

The hydrocarbon product 171 is combined with the second light hydrocarbon product 176a and then with the first light hydrocarbon product 166a and mixed in tank 182 to form a light hydrocarbon product 183. The light hydrocarbon product 183 is fed to the distillation column 184. Within the distillation column 184, the light hydrocarbon product 183 produced by the Fischer-Tropsch process 160 is distilled into naphtha products 186 and diesel products 187. Heavy hydrocarbons 185 produced in the distillation column 184 are removed from the distillation column 184 and fed to the hydrocracker 170. Purge gases 188 from the distillation column 184 may be combined with the tail gases 177 and fed to the hydrogen separation membrane 178 for separation of hydrogen from the purge gases 188.

The CTL process 100 may also include a heat recovery and power generation process 190. The heat recovery and power generation process 190 converts heat recovered from the CTL process 100 into power. Recovered steam 58, such as high pressure steam 56 and medium pressure steam 54, collected in the CTL process 100 is fed to a heat recovery steam generator 194 along with boiler feed water 52 and air 119. Tail gases 191 from the hydrogen separation membrane 178 are combined with air 119 and burned in a gas turbine 192 connected to a generator to produce electricity. Exhaust gases 193 from the gas turbine 192 are fed to the heat recovery steam generator 194 to further heat the boiler feed water 52 and recovered steam 58 fed to the heat recovery steam generator 194. Superheated steam 195 produced in the heat recovery steam generator 194 is fed to a condensing steam turbine 196 which produces electricity from the expansion and cooling of the superheated steam 195. The water produced in the condensing steam turbine 196 is fed to a pump 199 and pumped throughout the CTL process 100 to be used as boiler feed water 52.

The exhaust gases 193 are cooled in the heat recovery steam generator 194 and are then fed to a stack 198. The gases exiting the stack may be further treated or released into the environment.

Although CTL processes 100 such as the one illustrated in FIG. 1 may be used to produce liquid hydrocarbons and liquid fuel products from synthesis gas produced by the gasification of coal, such processes often produce a larger amount of pollutants than do conventional petroleum extraction and refining processes associated with liquid fuel production. One of the most prevalent pollutants produced by coal gasification is carbon dioxide (CO₂). Increased production of synthesis gases from coal gasification to supply Fischer-Tropsch processes with sufficient synthesis gas to produce liquid hydrocarbons and liquid fuel products would result in the unwanted increased production of carbon dioxide emissions. Proposals to counter the increased carbon dioxide emissions resulting from coal gasification have included proposals to sequester the carbon dioxide or otherwise convert the carbon dioxide produced in such a process prior to release into the environment. Carbon dioxide sequestration processes are expensive and add additional costs to liquid hydrocarbon production processes. Other proposed alternatives to encourage sequestration or conversion of carbon dioxide to a non-pollutant have also been made, including proposals to tax carbon dioxide emissions from coal gasification and Fischer-Tropsch processes.

Furthermore, coal gasification processes tend to be fairly inefficient. For example, a CTL process 100 such as that illustrated in FIG. 1 may convert about thirty-percent of the carbon contained in the coal fed to the process to liquid hydrocarbons or liquid fuel products. The remaining seventy-percent of the carbon in the coal is converted to carbon dioxide, other pollutants, or solid wastes that must be disposed.

Therefore, it would be desirable to improve the efficiency of coal-to-liquid hydrocarbon production process and to increase the conversion of carbon obtained from coal to liquid fuel products. It would also be desirable to decrease the carbon dioxide emissions of coal-to-liquid hydrocarbon production processes.

### BRIEF SUMMARY OF THE INVENTION

According to embodiments of the invention, the conversion of carbon to liquid fuel products in a coal-to-liquid hydrocarbon production process may be improved by modifying a conventional coal-to-liquid hydrocarbon production process or by incorporating the modifications into new process plants. The modifications to a coal-to-liquid hydrocarbon production process facilitate the conversion of carbon to liquid fuel products and reduce the amount of carbon-based pollutants produced by a coal-to-liquid hydrocarbon production process.

In some embodiments of the invention, a water-splitting process, such as an electrolysis or thermochemical process, for producing oxygen may be integrated with a liquid fuel production process, such as a coal-to-liquid hydrocarbon production process, or may be configured to supply oxygen to a liquid fuel production process. The oxygen may be used in the liquid fuel production process to gasify coal in the production of a synthesis gas containing hydrogen and carbon monoxide. The water-splitting process may produce oxygen from water utilizing conventional electrolysis processes. The power or heat required by the water-splitting process to produce oxygen from water may be supplied from a nuclear power source. The use of a nuclear power source to produce electricity or power to supply to the water-splitting process decreases the production of pollutants associated with conventional coal-fired electricity generation operations. The resulting liquid fuel production process therefore produces fewer pollutants. In addition, the use of a water-splitting process reduces the need for air separation units conventionally used with gasification processes to produce oxygen, reducing the equipment and operating costs associated with the production of liquid fuels, such as by the gasification of coal.

In other embodiments of the invention, the water-splitting process may also produce hydrogen. The water-splitting process may be configured to supply the produced hydrogen to the liquid fuel production process. In particular, hydrogen produced by the water-splitting process may be mixed with synthesis gas produced in the liquid fuel production process to achieve a desired ratio of hydrogen to carbon monoxide in the synthesis gas. The hydrogen produced by the water-splitting process may also be used to facilitate hydrocracking of heavy hydrocarbons or liquid fuel products produced from the synthesis gas generated in the liquid fuel production process.

The incorporation of a hydrogen-producing water-splitting process with a liquid fuel production process also allows for the reduction of equipment needed in a liquid fuel production process. The availability of hydrogen to mix with synthesis gas produced in the process reduces or eliminates the need for shift reactions within the liquid fuel production process. Therefore, equipment associated with such shift reactions may be made smaller or removed from the process, reducing the overall equipment and operational costs of the process.

According to still other embodiments of the invention, pollutant gases removed from the synthesis gases produced in a liquid fuel production process may be recycled to a coal gasification process or other synthesis gas production process. Carbon-containing compounds and pollutants, such as carbon dioxide, carbon monoxide, methane, alkanes, alkenes, alcohols, aldehydes, or other species, produced in a liquid fuel production process may be recycled to a coal gasification process, for example, where the carbon-containing pollutants may be further reacted to produce additional synthesis gas. The production of additional synthesis gas from the carbon-containing pollutants improves the yield of liquid hydrocarbons and liquid fuel products produced in the process while reducing the amount of carbon-containing pollutants produced by the process.

According to other particular embodiments of the invention, tail gases from a Fischer-Tropsch process associated with a liquid fuel production process, such as a coal-to-liquid hydrocarbon production process, may be recycled to generate additional synthesis gas in the process. In some embodiments, the tail gases may be recycled because they are not needed to supply energy due to the availability of an alternative energy source, such as a nuclear reactor.

The liquid fuel production processes of various embodiments of the invention provide enhanced yields of liquid hydrocarbons and liquid fuel products per ton of coal or fuel when compared to conventional processes. In addition, the liquid fuel production processes of embodiments of the invention reduce the production of carbon-containing pollutants in the process.

### BRIEF DESCRIPTION OF THE DRAWINGS

While the specification concludes with claims particularly pointing out and distinctly claiming that which is regarded as the present invention, this invention can be more readily understood and appreciated by one of ordinary skill in the art from the following description of the invention when read in conjunction with the accompanying drawings in which:

FIG. 1 illustrates a process flow and system diagram of a conventional coal-to-liquid hydrocarbon production process;

FIG. 2 illustrates a process flow and system diagram of a coal-to-liquid hydrocarbon production process according to certain embodiments of the invention;

FIG. 3 illustrates a process flow and system diagram of a coal-to-liquid hydrocarbon production process according to certain embodiments of the invention;

FIG. 4 illustrates a process flow and system diagram of a coal-to-liquid hydrocarbon production process according to certain embodiments of the invention; and

FIG. 5 illustrates a process flow and system diagram of a coal-to-liquid hydrocarbon production process according to certain embodiments of the invention.

### DETAILED DESCRIPTION OF THE ILLUSTRATED EMBODIMENTS

According to embodiments of the invention, a nuclear power source may be integrated with or incorporated into a liquid fuel production process, such as a coal-to-liquid hydrocarbon production process, to facilitate the production of hydrocarbons and liquid fuel products from coal. Although the additional heat produced by a nuclear power source may not be needed in a conventional coal-to-liquid hydrocarbon production process, the integration of a nuclear power source with a coal-to-liquid hydrocarbon production process, or other liquid fuel production process, facilitates the use of alternative processes to produce oxygen (O₂) and hydrogen (H₂), allowing expensive equipment to be removed from a liquid fuel production process.

While various embodiments of the invention may be integrated with liquid fuel production processes, many of the embodiments will be described with respect to a coal-to-liquid hydrocarbon production process. It is understood that other liquid fuel production processes may be substituted for the coal-to-liquid hydrocarbon production processes described with respect to particular embodiments of the invention.

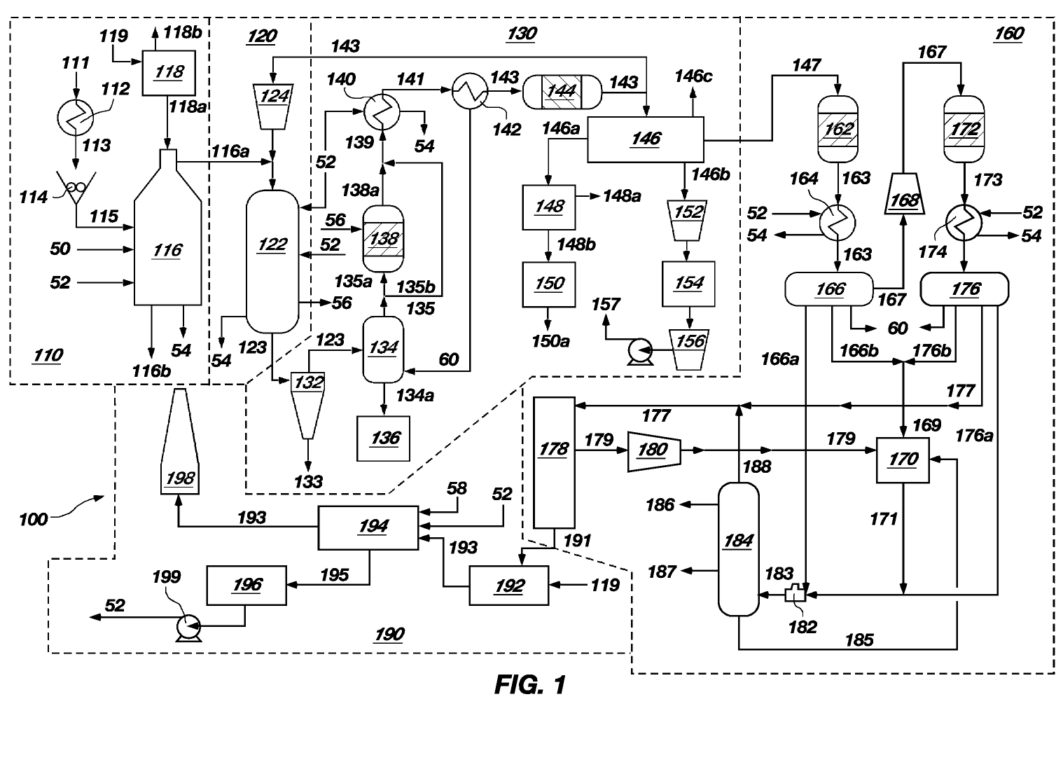

According to certain embodiments of the invention, a nuclear power source may be configured to provide electric power, heat, or a combination of electric power and heat to operate a water-splitting process. The water-splitting process may be used to generate oxygen and hydrogen from water. Oxygen produced by the water-splitting process may be substituted for the oxygen produced by an air separation unit 118 in a conventional CTL process 100 such as the process illustrated in FIG. 1. The alternative source of oxygen provided by the water-splitting process and nuclear power source eliminates the need for an air separation unit in a CTL process 100. Air separation units, such as air separation unit 118 in the conventional CTL process 100 illustrated in FIG. 1, are often expensive to install and expensive to operate. The use of oxygen from a water-splitting process operated with nuclear power may therefore decrease the costs associated with the operation of a coal-to-liquid hydrocarbon production process.

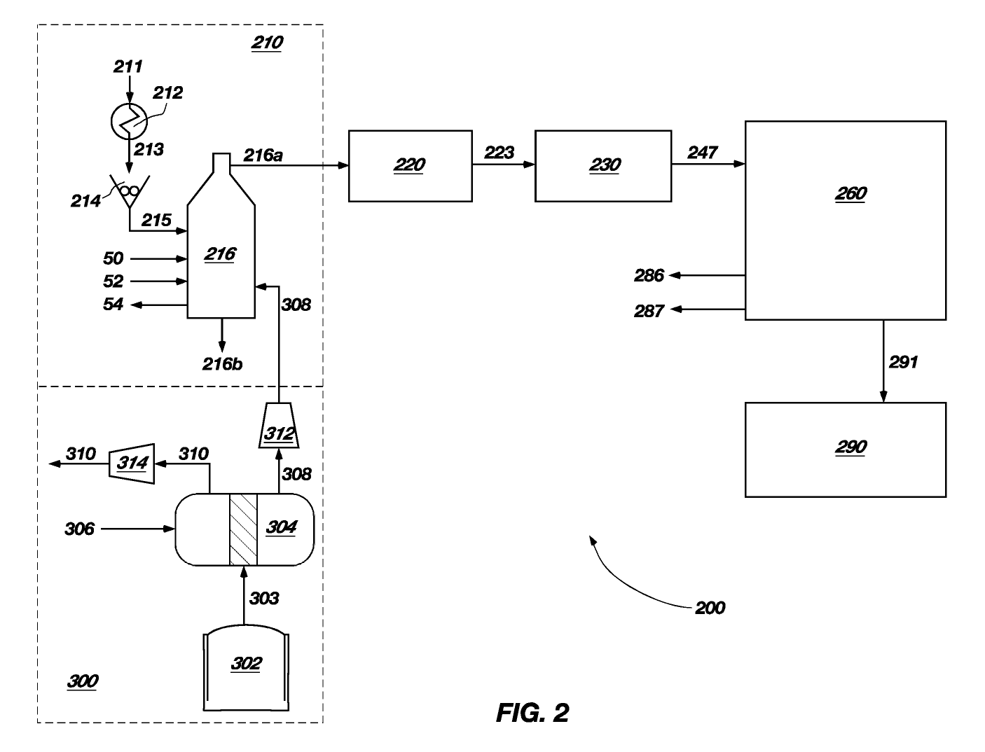

An example of a coal-to-liquid hydrocarbon production process 200 utilizing oxygen produced by a water-splitting process 300 rather than by an air separation unit is illustrated in FIG. 2. As with a conventional CTL process 100, coal-to-liquid hydrocarbon production processes 200 may include sub-processes such as a coal gasification process 210, a gas and heat recovery process 220, a gas cleanup process 230, a Fischer-Tropsch process 260, and a heat recovery and power generation process 290. The sub-processes of the coal-to-liquid hydrocarbon production process 200 which do not differ from convention CTL process 100 sub-processes are illustrated as sub-process blocks in FIG. 2.

The coal-to-liquid hydrocarbon production process 200 illustrated in FIG. 2 may also include a water-splitting process 300. For example, a water-splitting process 300 may include an electrolysis process or a thermochemical water-splitting process. While various water-splitting processes may be used with various embodiments of the invention, certain embodiments are described with respect to the use of an electrolysis process. It is understood that other water-splitting processes may be used in place of or in combination with an electrolysis process according to various embodiments of the invention. For example, a water-splitting process 300 according to embodiments of the invention may include one or more nuclear power sources 302, one or more electrolyzers 304, one or more oxygen compressors 312, and one or more hydrogen compressors 314.

The one or more nuclear power sources 302 may include any source of power resulting from nuclear energy. For example, multiple high-temperature nuclear reactors may be used to produce electricity 303 capable of operating one or more electrolyzers 304 to produce oxygen 308 and hydrogen 310 from water 306. The oxygen 308 may be compressed by one or more compressors 312 and fed to the entrained flow gasifier 216 of the coal gasification process 210. The introduction of oxygen 308 from the electrolyzers 304 into the entrained flow gasifier 216 eliminates the need for an oxygen supply from an air separation unit.

Electrolyzers 304 may include conventional electrolyzers 304 such as electrolyzers used in conventional low temperature electrolysis, high temperature electrolysis, or thermochemical processes.

According to particular embodiments of the invention, coal 211 for use in the coal gasification process 210 may be fed to one or more dryers 212 to reduce moisture in the coal 211. The one or more dryers 212 may include heat exchangers or other conventional drying processes. The dried coal 213 may be fed to a pulverizer 214 where the dried coal 213 is pulverized, crushed, or otherwise reduced to a sufficient size for coal gasification. The pulverized coal 215 may be fed to an entrained flow gasifier 216 to gasify the pulverized coal 215. Oxygen 308 or compressed oxygen 308 from the electrolysis process 300 is also fed to the entrained flow gasifier 216 to facilitate gasification of coal within the entrained flow gasifier 216. Steam 50 and boiler feed water 52 may also be fed to the entrained flow gasifier 216. The gasification of coal within the entrained flow gasifier 216 produces a synthesis gas product 216a, or syngas, comprising hydrogen and carbon monoxide. Slag 216b produced in the entrained flow gasifier 216 may be removed from the entrained flow gasifier 216 and disposed of as desired. Heat generated in the entrained flow gasifier 216 may be used to produce medium pressure steam 54 from the boiler feed water 52 introduced to the entrained flow gasifier 216. The medium pressure steam 54 produced by the entrained flow gasifier 216 may be used elsewhere in the coal-to-liquid hydrocarbon production process 200 as desired.

While the coal gasification processes 210 of the coal-to-liquid hydrocarbon production processes 200 described in various embodiments of the invention utilize entrained flow gasifiers 216 to gasify coal, it is understood that other gasification units may be substituted for the entrained flow gasifiers 216. For example, gasification units that may be used with coal gasification processes 210 of the various embodiments of the invention may include, but are not limited to, entrained flow gasifiers, counter-current fixed bed gasifiers, co-current fixed bed gasifiers, and fluid bed gasifiers. Other coal gasification equipment capable of producing a synthesis gas from the gasification of coal could also be used as a gasification unit.

The entrained flow gasifier 216, or other gasification equipment, may be configured to gasify coal at a desired temperature. In certain embodiments, for example, the entrained flow gasifier 216 may operate within a temperature range of between about 1300° C. and about 1600° C. Operation within such a temperature range may reduce the formation of carbon dioxide (CO₂) and methane (CH₄) within the entrained flow gasifier 216.

The synthesis gas product 216a from the coal gasification process 210 may be fed to a gas and heat recovery process 220 which produces a synthesis gas product 223 that may be fed to a gas cleanup process 230. The gas cleanup process 230 removes pollutants such as hydrogen sulfide (H₂S) and carbon dioxide (CO₂) from the synthesis gas product 223. For example, processes such as Rectisol processes, Claus processes, SCOT processes, and TEG dehydration processes may be used to remove pollutants from the synthesis gas product 223. The cleaned synthesis gas from the gas cleanup process 230 may be fed to a Fischer-Tropsch process 260 as a syngas product stream 247. The Fischer-Tropsch process 260 may convert the syngas product stream 247 into naphtha products 286 and diesel products 287. Tail gases 291 from the Fischer-Tropsch process 260 may be fed to a heat recovery and power generation process 290 to produce electricity.

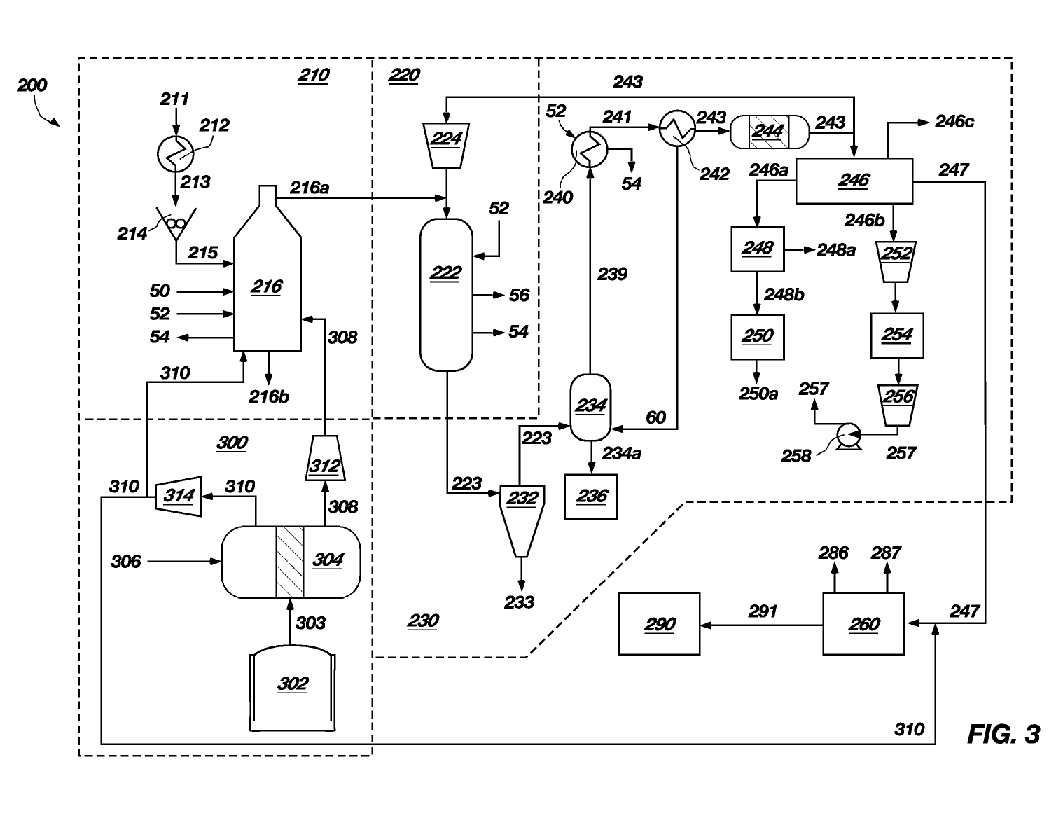

According to other embodiments of the invention, hydrogen produced by a water-splitting process 300 in a coal-to-liquid hydrocarbon production process 200 may be added to synthesis gas produced in the coal-to-liquid hydrocarbon production process 200. As illustrated in FIG. 3, hydrogen 310 produced by one or more electrolyzers 304 in a water-splitting process 300 may be compressed by one or more compressors 314 and combined with a synthesis gas product stream 247 being fed to a Fischer-Tropsch process 260 of a coal-to-liquid hydrocarbon production process 200. The addition of hydrogen 310 to the synthesis gas product stream 247 may be used to obtain a desired ratio of hydrogen to carbon monoxide within the synthesis gas product stream 247. For example, in some coal-to-liquid hydrocarbon production processes 200 according to embodiments of the invention, the synthesis gas fed to the Fischer-Tropsch process 260 may have a hydrogen to carbon monoxide mole ratio (H₂/CO) of about 2.15. The ability to supplement the synthesis gas product stream 247 with hydrogen from the water-splitting process 300 allows the H₂/CO ratio of the Fischer-Tropsch process 260 feed gas to be tailored or configured as desired.

The addition of hydrogen 310 to the synthesis gas product stream 247 may also reduce or eliminate the need for water-gas shift chemistry in a gas cleanup process 230, which removes the need for the inclusion of a sour shift reactor in the coal-to-liquid hydrocarbon production process 200. Instead of including equipment within the coal-to-liquid hydrocarbon production process 200 to shift the ratio of hydrogen to carbon monoxide in a synthesis gas product stream 247, hydrogen 310 produced in a water-splitting process 300 may be combined with the synthesis gas product stream 247 to achieve the desired ratios of hydrogen and carbon dioxide for a Fischer-Tropsch process 260.

One configuration for particular embodiments of the invention is illustrated in FIG. 3. A synthesis gas product 216a from the coal gasification process 210 may be introduced into a syngas cooler 222 of a gas and heat recovery process 220 of the coal-to-liquid hydrocarbon production process 200. The syngas cooler 222 cools the hot synthesis gas product 216a and produces a cool synthesis gas product 223 which is withdrawn from the syngas cooler 222 and fed to the gas cleanup process 230. Boiler feed water 52 fed to the syngas cooler 222 may absorb heat from the hot synthesis gas product 216a fed to the syngas cooler 222, resulting in the production of high pressure steam 56 and medium pressure steam 54 which are removed from the syngas cooler 222 for use elsewhere in the coal-to-liquid hydrocarbon production process 200.

The syngas cooler 222 may include conventional equipment for cooling gases produced in a coal gasification process 210. For example, the syngas cooler 222 may include one or more convective syngas coolers, radiant syngas coolers, or a combination of convective and radiant syngas coolers.

The synthesis gas product 223 may be fed to a gas cleanup process 230. The gas cleanup process may include a cyclone 232, a water scrubber 234, a black water treatment system 236, a heat exchanger 240, a condenser 242, an activated carbon bed 244, a Rectisol process 246, a Claus process 248, a SCOT process 250, a TEG dehydration process 254, and one or more compressors 252, 256 associated with the TEG dehydration process 254. While the gas cleanup process 230 illustrated in FIG. 3 includes single pieces of equipment utilized in the process, multiple pieces of equipment may also be used. For example, the heat exchanger 240 may be replaced by two or more heat exchangers 240 as desired. Scaling of the gas cleanup process 230 in order to accommodate desired product flows is feasible.

Fly ash 233 and other particulates entrained in the synthesis gas product 223 fed to the cyclone 232 may be removed from the cyclone 232 and the synthesis gas product may be fed to a water scrubber 234. Water 60 fed to the water scrubber 234 removes pollutants and other impurities from the synthesis gas product 223 and discharges the water 60 and other impurities as waste water stream 234a which may be fed to a black water treatment system 236 or other treatment system to treat the waste water stream 234a. A scrubbed synthesis gas product 239 from the water scrubber 234 may be fed to a heat exchanger 240 to cool the scrubbed synthesis gas product 239. Boiler feed water 52 fed to the heat exchanger 240 may absorb heat from the scrubbed synthesis gas 239, producing steam such as a medium pressure steam 54 that may be used elsewhere in the coal-to-liquid hydrocarbon production process 200. The cooled, scrubbed synthesis gas product 241 may be fed to a condenser 242 where water 60 in the cooled, scrubbed synthesis gas product 241 is recovered. The synthesis gas product 243 exiting the condenser 242 may then be treated to further remove pollutants from the synthesis gas product 243.

Mercury in the synthesis gas product 243 may be removed by contacting the synthesis gas product 243 with activated carbon in an activated carbon bed 244. The synthesis gas product 243 exiting the activated carbon bed 244 may be fed to one or more processes for removing hydrogen sulfide and carbon dioxide from the synthesis gas product 243. In addition, a portion of the synthesis gas product 243 may be diverted to a compressor 224 and then mixed with synthesis gas product 216a from the coal gasification process 210. In some embodiments, a portion of the synthesis gas product 243 exiting the condenser 242 may be diverted to a compressor 224 rather than being fed to the activated carbon bed 244. This diversion may be in combination with or in place of the diversion of the portion of synthesis gas product 243 exiting the activated carbon bed 244.

As illustrated in FIG. 3, at least a portion of the synthesis gas product 243 may be fed to a Rectisol process 246 to remove hydrogen sulfide (H₂S) and carbon dioxide (CO₂) from the synthesis gas product 243. The Rectisol process 246 may produce a first product stream 246a containing hydrogen sulfide and carbon dioxide, a second product stream 246b containing carbon dioxide, a purge gas 246c, and a synthesis gas product stream 247.

The first product stream 246a may be further treated by a Claus process 248 and a SCOT process 250 to remove sulfur and carbon dioxide pollutants from the first product stream 246a and to produce tail gases 250a that are sufficiently clean to be released into the environment. A sulfur product 248a may be discharged from the Claus process 248.

The second product stream 246b may be compressed in compressor 252 and treated with a TEG dehydration process 254 to purify carbon dioxide from the second product stream 246b. Carbon dioxide gas removed from the second product stream 246b may be fed to a compressor 256 and cooled to form liquid carbon dioxide 257. The liquid carbon dioxide 257 may be pumped with a pump 258 to a storage container or other process as desired. Thus, carbon dioxide pollutants recovered from the synthesis gas product 243 may be converted to liquid carbon dioxide for use or sale.

The purge gas 246c from the Rectisol process 246 may be released into the environment. Rectisol processes 246, Claus processes 248, SCOT processes 250 and TEG dehydration processes 254 are well known processes and conventional equipment may be readily utilized to carry out such processes in the gas cleanup process 230 of the coal-to-liquid hydrocarbon production processes 200 according to embodiments of the invention may be readily utilized.

The synthesis gas product stream 247 produced by the gas cleanup process 230 may be fed to one or more Fischer-Tropsch processes 260 where the synthesis gas product stream 247 is converted into naphtha products 286 and diesel products 287. According to embodiments of the invention, hydrogen 310 from a water-splitting process 300 may be mixed with the synthesis gas product stream 247 prior to or at the same time that the synthesis gas product stream 247 is fed to the Fischer-Tropsch process 260. The addition of hydrogen 310 to the synthesis gas product stream 247 may be controlled to achieve a desired ratio of hydrogen and carbon monoxide being fed to the Fischer-Tropsch process 260.

Tail gases 291 from the Fischer-Tropsch process 260 may be fed to a heat recovery and power generation process 290 if desired.

Although the coal-to-liquid hydrocarbon production process 200 illustrated in FIG. 3 includes the use of both the hydrogen 310 and oxygen 308 produced by the water-splitting process 300 in the coal-to-liquid hydrocarbon production process 200, it is understood that various embodiments of the invention may also utilize just the oxygen 308 produced by the electrolyzers 304 as a feed stream to the coal gasification process 210 as illustrated in FIG. 2, or just the hydrogen 310 produced by the electrolyzers 304 as a synthesis gas product stream 247 additive.

In still other embodiments of the invention, hydrogen 310 produced by a water-splitting process 300 may be fed to a Fischer-Tropsch process 260 and combined with hydrogen produced by a hydrogen membrane of the Fischer-Tropsch process 260 (not shown). For example, hydrogen 310 produced by the water-splitting process 300 may be used with hydrocracking and hydrotreating processes of a Fischer-Tropsch process 260.

In other embodiments of the invention, hydrogen 310 produced by a water-splitting process 300 may be fed to a gasifier 216 as illustrated in FIG. 3. Hydrogen 310 fed to a gasifier 216 may assist in the conversion of carbon dioxide to carbon monoxide when carbon dioxide is formed in the gasifier 216 or recycled to the gasifier 216.

The combination of a water-splitting process 300 with a coal-to-liquid hydrocarbon production process 200 as illustrated in FIGS. 2 and 3 offers many advantages over conventional coal-to-liquid hydrocarbon production processes. For example, as illustrated in FIG. 2, the use of oxygen 308 produced by the water-splitting process 300 with the coal gasification process 210 may reduce or eliminate the need for an air separation unit to produce oxygen for coal gasification. In other instances, the hydrogen 310 produced in the water-splitting process 300 may be used to control the ratios of hydrogen and carbon monoxide in a synthesis gas product stream 247 fed to a Fischer-Tropsch process 260. The ability to balance or shift the ratios of hydrogen and carbon monoxide in the synthesis gas product stream 247 with hydrogen 310 produced by the water-splitting process 300 reduces or eliminates the need for sour shift reaction equipment in the coal-to-liquid hydrocarbon production process 200. The hydrogen 310 may also be utilized to facilitate hydrocracking and hydrotreating processes within a Fischer-Tropsch process 260. Thus, the use of oxygen 308, hydrogen 310, or a combination of oxygen 308 and hydrogen 310 produced by a water-splitting process 300 with a coal-to-liquid hydrocarbon production process 200 may reduce the amount of equipment necessary to operate the coal-to-liquid hydrocarbon production process 200 and reduce costs associated with the operation of the processes of the coal-to-liquid hydrocarbon production process 200.

The combination of a water-splitting process 300 with a coal-to-liquid hydrocarbon production process 200 as illustrated in FIGS. 2 and 3 may also improve the production of liquid hydrocarbons and liquid fuel products and reduce the amount of pollutants generated in the coal-to-liquid hydrocarbon production process 200. For example, simulations of the conventional CTL process 100 illustrated in FIG. 1 were run and compared to simulations of the coal-to-liquid hydrocarbon production process 200 illustrated in FIG. 3. The simulations were performed using Aspen™ software and the results are illustrated in Table 1:

TABLE 1

| | CTL Process 100 | CTL Process 200 |
|---|---|---|
| | (FIG. 1) | (FIG. 3) |
| Coal Feed (ton/day) | 18,840 | 18,840 |
| Liquid Fuel Produced | 26,037 | 58,182 |
| (bbl/day) | | |
| Conversion | 1.38 | 3.09 |
| (bbl liquids/ton of coal) | | |
| Carbon Partitioned to Liquid | 29.5 | 65.8 |
| Fuel (% of carbon input) | | |

The data of Table 1 indicate that the coal-to-liquid hydrocarbon production process 200 according to embodiments of the invention produces more liquid fuel per ton of coal than does a conventional CTL process 100. The increased production of liquid fuel from the coal in a coal-to-liquid hydrocarbon production process 200 also increases the amount of carbon converted to liquid fuel, which in turn results in less carbon in the process which can be converted to carbon dioxide pollutants. Thus, the use of oxygen 308 and hydrogen 310 produced by a water-splitting process 300 with a coal-to-liquid hydrocarbon production process 200 according to embodiments of the invention provides improved production of liquid hydrocarbon fuels over conventional carbon-to-liquid hydrocarbon production processes and reduces carbon-based pollutants produced in such processes.

According to still other embodiments of the invention, carbon recovery in a coal-to-liquid hydrocarbon production process 200 may be improved by recycling carbon-containing gas streams in the coal-to-liquid hydrocarbon production process 200 to the coal gasification process 210.

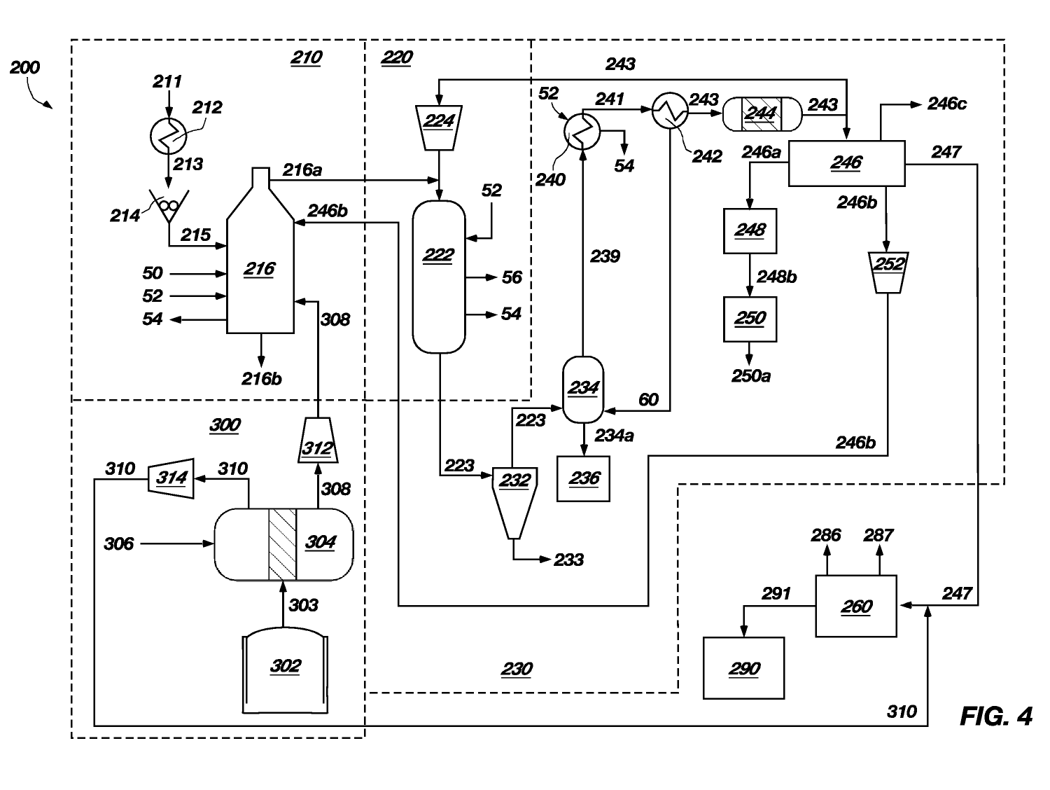

For example, in certain embodiments of the invention, carbon dioxide gases separated from the synthesis gas product 243 fed to a Rectisol process 246 may be recycled to the entrained flow gasifier 216. The introduction of additional carbon dioxide into the entrained flow gasifier 216 may result in additional reactions of the carbon dioxide with hydrogen to produce additional carbon monoxide. An example of such a process is illustrated in FIG. 4.

The second product stream 246b produced in the Rectisol process 246 contains carbon dioxide. As illustrated in FIG. 4, the second product stream 246b may be compressed in compressor 252 and recycled to the entrained flow gasifier 216, or other gasification equipment, so that the carbon dioxide in the second product stream 246b may react with hydrogen in the entrained flow gasifier 216 to produce carbon monoxide.

The ability to redirect the second product stream 246b from the Rectisol process 246 in the coal-to-liquid hydrocarbon production process 200 illustrated in FIG. 4 is achieved in part because of the integration of the water-splitting process 300 with the coal-to-liquid hydrocarbon production process 200. The elimination of a sour shift reactor from the coal-to-liquid hydrocarbon production process 200 due to the availability of hydrogen 310 from the water-splitting process 300 reduces the size of the second product stream 246b from the Rectisol process 246. The reduction in the size of the second product stream 246b allows the second product stream 246b to be recycled to the coal gasification process 210. Recycling of the carbon containing second product stream 246b improves carbon conversion in the coal-to-liquid hydrocarbon production process 200 and decreases the amount of carbon-based pollutants produced by the process.

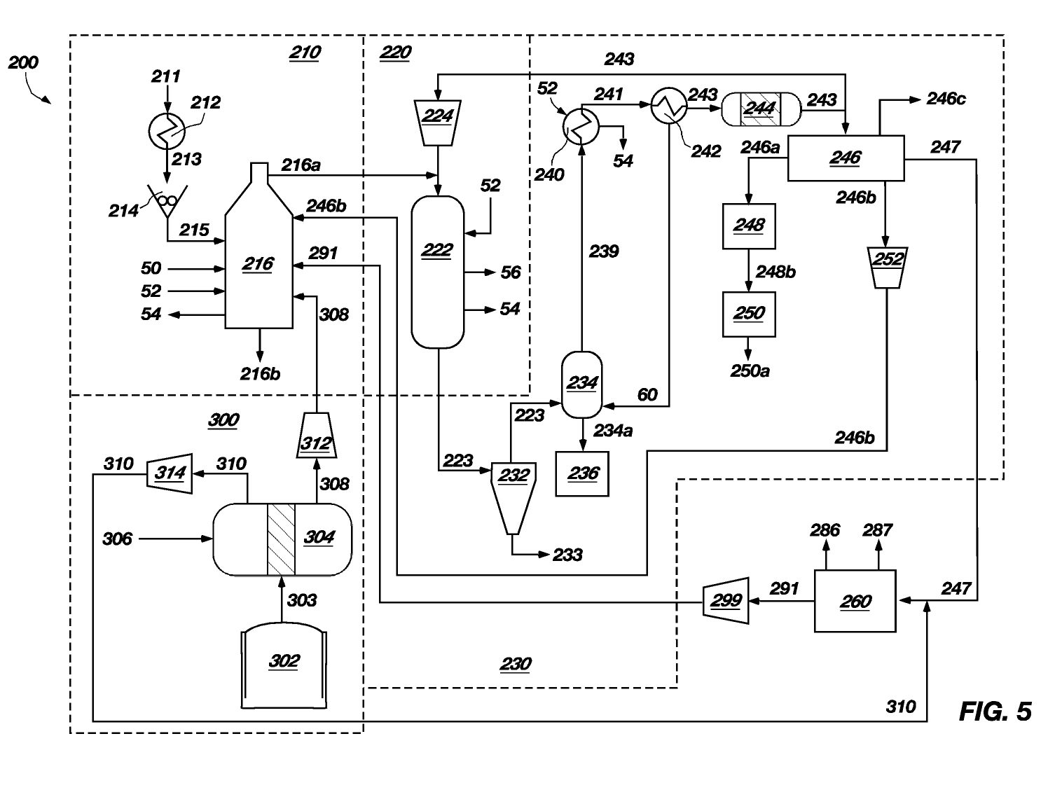

In still other embodiments of the invention, tail gases 291, light fuel components, or a mixture of tail gases 291 and light fuel components from a Fischer-Tropsch process 260 of a coal-to-liquid hydrocarbon production process 200 may be recycled to the coal gasification process 210. In particular, carbon-containing tail gases 291 and any light fuel components may be fed to the entrained flow gasifier 216, or other gasification unit, to facilitate the conversion of carbon in a feedstock to the entrained flow gasifier 216 to desirable fuel products. An example of such recycling is illustrated in FIG. 5.

As illustrated in FIG. 5, the tail gases 291 from a Fischer-Tropsch process 260 are recycled to the entrained flow gasifier 216. The tail gases 291 may undergo compression in compressor 299 before being fed to the entrained flow gasifier 216. The recycling of the tail gases 291 to the coal gasification process 210 eliminates the tail gas 291 feed for a heat recovery and power generation process 290 in the coal-to-liquid hydrocarbon production process 200. The elimination of the tail gas 291 feed to a heat recovery and power generation process 290 eliminates the need for the heat recovery and generation equipment associated with such processes. In addition, recycling of carbon-containing tail gases 291, and in some instances light hydrocarbons, from the Fischer-Tropsch process 260 to the coal gasification process 210 facilitates further conversion of the tail gases 291 into synthesis gas. For example, the tail gases 291 may include light hydrocarbons, residual hydrogen, carbon monoxide, water, and carbon dioxide that may be converted into synthesis gas in the coal gasification process 210.

While the process illustrated in FIG. 5 includes the recycling of the tail gases 291 from the Fischer-Tropsch process 260 and the second product stream 246b from the Rectisol process 246 to the entrained flow gasifier 216, it is understood that the tail gases 291 from the Fischer-Tropsch process 260 may be recycled without recycling the second product stream 246b to the entrained flow gasifier 216. In addition, it is understood that the recycled tail gases 291 from the Fischer-Tropsch process 260 may include all or only a portion of the total tail gases 291 produced by the Fischer-Tropsch process 260. Similarly, the second product stream 246b from the Rectisol process 246 that is recycled to the entrained flow gasifier 216 may include all or only a portion of the second product stream 246b.

It is also understood that the recycling of the second product stream 246b, the tail gases 291, or a portion or all of the second product stream 246b and tail gases 291 to the entrained flow gasifier 216 may occur in a process that does not employ a water-splitting process 300. For example, in process 100 of FIG. 1, carbon-containing products from the gas cleanup process 130 and from the Fischer-Tropsch process 160 may be recycled to the entrained flow gasifier 116 in a manner similar to that illustrated in FIGS. 4 and 5.

A simulated comparison of the coal-to-liquid hydrocarbon production process 200 illustrated in FIG. 5 with the coal-to-liquid hydrocarbon production process 200 illustrated in FIG. 3 and with the conventional CTL process 100 illustrated in FIG. 1 was performed using Aspen™ software. In addition, a smaller coal-to-liquid hydrocarbon production process 200 based upon the configuration illustrated in FIG. 5 was simulated to compare with larger process configurations. The results of the simulations are given in Table 2:

TABLE 2

| | CTL | CTL | | |
|---|---|---|---|---|
| | Process | Process | CTL | Small |
| | 100 | 200 | Process 200 | CTL |
| | (FIG. 1) | (FIG. 3) | (FIG. 5) | Process |
| Coal Feed | 18,800 | 18,800 | 18,800 | 5,800 |
| (ton/day) | | | | |
| Liquid Fuel Produced | 26,000 | 58,200 | 84,672 | 26,000 |
| (bbl/day) | | | | |
| Conversion | 1.38 | 3.09 | 4.49 | 4.49 |
| (bbl liquids/ton coal) | | | | |
| Yield of Liquid Fuel | 29.5 | 65.8 | 95.7 | 95.7 |
| (% of carbon input) | | | | |

The data in Table 2 confirm that the coal-to-liquid hydrocarbon production processes 200 illustrated in FIGS. 3 and 5 produce a greater amount of liquid hydrocarbon fuels for a given amount of coal than does the conventional CTL process 100 illustrated in FIG. 1. In addition, the coal-to-liquid hydrocarbon production process 200 illustrated in FIG. 5 produces a greater amount of liquid hydrocarbon fuel as compared to the coal-to-liquid hydrocarbon production process 200 illustrated in FIG. 3 for an equivalent amount of coal. The data also indicate that the yield of liquid hydrocarbon fuels produced as a percentage of carbon input into the processes is greatest in the coal-to-liquid hydrocarbon production process 200 illustrated in FIG. 5. The coal-to-liquid hydrocarbon production process 200 illustrated in FIG. 3 is also better at converting carbon to liquid hydrocarbon fuels than is the conventional CTL process 100 illustrated in FIG. 1.

The data of Table 2 also provide some insight into the production efficiencies of certain embodiments of the invention. The small coal-to-liquid hydrocarbon production process simulated for Table 2 produces the same amount of liquid hydrocarbon fuels as does the conventional CTL process illustrated in FIG. 1. However, the small coal-to-liquid hydrocarbon production process based upon the configuration of the coal-to-liquid hydrocarbon production process 200 illustrated in FIG. 5 produces the same amount of liquid hydrocarbon fuels as does the conventional CTL process 100 with 5,800 tons of coal per day as compared to the 18,800 tons per day required for the conventional CTL process 100. As a result, coal-to-liquid hydrocarbon production processes 200 according to embodiments of the invention are much more efficient at converting coal to hydrocarbon fuels than are conventional processes.

The data in Table 2 also illustrate the fact that coal-to-liquid hydrocarbon production processes according to particular embodiments of the invention have reduced pollutant emissions as compared to conventional CTL processes 100. The increased yield of liquid fuel based upon the carbon input into each process indicates that the processes illustrated in FIGS. 3 and 5 convert more carbon to liquid fuel than does a conventional process, resulting in less carbon-based pollution from the coal-to-liquid hydrocarbon production process.

While various embodiments of the invention employ the use of a Fischer-Tropsch process 260 to produce liquid hydrocarbons from a synthesis gas, it is understood that other processes may be used in combination with, or as a substitute for, the Fischer-Tropsch process 260 to produce liquid hydrocarbons, liquid fuel products, or a combination of liquid hydrocarbons and liquid fuel products. For example, any processes capable of converting a synthesis gas to liquid hydrocarbons or liquid fuel products may be used in place of, or in combination with, the Fischer-Tropsch process 260. In particular, processes having preferred hydrogen to carbon monoxide ratios may be combined with various embodiments of the invention to produce liquid hydrocarbons and liquid fuel products according to the desired ratios. In addition, synthesis gas conversion processes which produce carbon-containing purge gases may be integrated with various embodiments of the invention to recycle the purge gases to produce additional synthesis gas for conversion to liquid hydrocarbons and liquid fuel products. Examples of processes that may be substituted for, or combined with, a Fischer-Tropsch process 260 include, but are not limited to, processes for producing methanol or dimethyl ether.

In addition, while various embodiments of the invention are described with respect to the production of liquid hydrocarbons, it is understood that other liquid fuel products may also be produced using such embodiments.

The integration of a water-splitting process 300 with a coal-to-liquid hydrocarbon production process 200 according to embodiments of the invention improves the conversion of carbon in coal to liquid hydrocarbon fuels while also reducing the total amount of carbon pollutants produced in such processes.

While the water-splitting processes 300 of the present invention include the use of nuclear power sources 302 to provide heat or electricity for the water-splitting process 300, other power supplies may also be used to operate the electrolyzers 304. For example, nuclear power sources 302 may be substituted or combined with wind power, hydroelectric power, geothermal power, tidal power, or solar power to produce sufficient energy to operate the water-splitting process 300. In addition, conventional coal-fired or combustion power plants may be used to supply power to the electrolyzers 304 to generate hydrogen and oxygen for use with coal-to-liquid hydrocarbon production processes 200 according to embodiments of the invention.

The various coal-to-liquid hydrocarbon production processes 200 according to embodiments of the invention may also be scaled up or scaled down to achieve a desired coal consumption rate or hydrocarbon production rate. In addition, alternative fuels, such as carbon-containing fuels, may be used or burned in the coal-to-liquid hydrocarbon production processes 200 to produce synthesis gases. For example, fuels that may be used include, but are not limited to, coal, oil shale, biomass, refuse, waste materials, natural gas, lignite, and mixtures thereof.

Having thus described certain embodiments of the present invention, it is understood that the invention defined by the appended claims is not to be limited by particular details set forth in the above description, as many apparent variations thereof are contemplated without departing from the spirit or scope thereof as hereinafter claimed.

What is claimed is:

1. A liquid fuel production system, comprising:
at least one gasification process apparatus configured to produce a synthesis gas;
at least one synthesis gas conversion process apparatus configured to receive a mixture of the synthesis gas; and
at least one recycle stream configured to remove a carbon-containing tail gas from the at least one synthesis gas conversion process apparatus and feed the carbon-containing tail gas to the at least one gasification process apparatus.

2. The liquid fuel production system of claim 1, wherein the at least one gasification process apparatus comprises a gasification unit selected from the group consisting of an entrained flow gasifier, a counter-current fixed bed gasifier, a co-current fixed bed gasifier, and a fluid bed gasifier.

3. The liquid fuel production system of claim 1, wherein the at least one gasification process apparatus comprises at least one gasification process apparatus configured to receive a carbon-containing tail gas feed from the at least one synthesis gas conversion process apparatus.

4. The liquid fuel production system of claim 1, wherein the at least one synthesis gas conversion process apparatus comprises a Fischer-Tropsch process apparatus.

5. The liquid fuel production system of claim 1, further comprising:
a gas cleanup process apparatus for removing pollutants from the synthesis gas; and
a pollutant recycle stream configured to remove a carbon-containing gas from the gas cleanup process apparatus and feed the carbon-containing gas to the at least one gasification process apparatus.

6. The liquid fuel production system of claim 1, further comprising a water-splitting process apparatus configured to produce oxygen and hydrogen and to feed at least a portion of the hydrogen to the at least one synthesis gas conversion process.

7. The liquid fuel production system of claim 6, wherein the water-splitting process apparatus comprises an electrolysis process apparatus powered by a power source selected from the group consisting of nuclear power, wind power, geothermal power, tidal power, hydroelectric power, and solar power.

8. The liquid fuel production system of claim 6, wherein the water-splitting process apparatus comprises a thermochemical water-splitting process apparatus.

9. A liquid fuel production system, comprising:
at least one gasification process apparatus configured to produce a synthesis gas;
at least one synthesis gas conversion process apparatus configured to receive a mixture of the synthesis gas;
at least one gas cleanup process apparatus for removing pollutants from the synthesis gas; and
at least one pollutant recycle stream configured to remove a carbon-containing gas from at least one gas cleanup process apparatus and to feed the carbon-containing gas to the at least one gasification process apparatus.

10. The liquid fuel production system of claim 9, wherein the at least one gasification process apparatus comprises a gasification unit selected from the group consisting of an entrained flow gasifier, a counter-current fixed bed gasifier, a co-current fixed bed gasifier, and a fluid bed gasifier.

11. The liquid fuel production system of claim 9, wherein the at least one gasification process apparatus comprises at least one gasification process apparatus configured to receive the at least one pollutant recycle stream from the at least one gas cleanup process apparatus.

12. The liquid fuel production system of claim 9, further comprising a water-splitting process apparatus configured to produce oxygen and hydrogen and to feed at least a portion of the hydrogen to the at least one synthesis gas conversion process.

13. The liquid fuel production system of claim 12, wherein the water-splitting process apparatus comprises an electrolysis process apparatus powered by a power source selected from the group consisting of nuclear power, wind power, geothermal power, tidal power, hydroelectric power, and solar power.

14. The liquid fuel production system of claim 12, wherein the water-splitting process apparatus comprises a thermochemical water-splitting process apparatus.

15. A method for converting a carbon-containing fuel to liquid fuel, comprising:
providing a carbon-containing fuel supply;
providing a carbon-containing gas supply;
gasifying at least a portion of the carbon-containing fuel supply and the carbon-containing gas supply to produce a synthesis gas; and
converting the synthesis gas into liquid hydrocarbons using a synthesis gas conversion process.

16. The method of claim 15, wherein providing a carbon-containing fuel supply comprises providing at least one carbon-containing fuel selected from the group consisting of coal, oil shale, biomass, refuse, waste materials, natural gas, lignite, and mixtures thereof.

17. The method of claim 15, wherein providing a carbon-containing gas supply further comprises providing carbon-containing tail gases from the synthesis gas conversion process.

18. The method of claim 15, wherein providing a carbon-containing gas supply further comprises:
providing a gas cleanup process;
feeding at least a portion of the produced synthesis gas to the gas cleanup process;
removing carbon-containing gas pollutants from the synthesis gas in the gas cleanup process; and
providing at least a portion of the carbon-containing gas pollutants as the carbon-containing gas supply.

19. The method of claim 15, wherein gasifying at least a portion of the carbon-containing fuel supply and the carbon-containing gas supply to produce a synthesis gas comprises gasifying the carbon-containing fuel supply and the carbon-containing gas supply in a gasifier selected from the group consisting of an entrained flow gasifier, a counter-current fixed bed gasifier, a co-current fixed bed gasifier, and a fluid bed gasifier.

20. The method of claim 15, further comprising:
providing a water-splitting process configured to produce oxygen and hydrogen; and
combining at least a portion of the hydrogen produced by the water-splitting process with the synthesis gas.
